(19) United States
(12) Patent Application Publication
(10) Pub. No.: US 2019/0060019 A1
(43) Pub. Date: Feb. 28, 2019

(54) FORCE ESTIMATION USING ROBOTIC MANIPULATOR FORCE TORQUE SENSORS

(71) Applicant: TransEnterix Surgical, Inc., Morrisville, NC (US)

(72) Inventor: Alexander John MARET, Apex, NC (US)

(73) Assignee: TransEnterix Surgical, Inc., Morrisville, NC (US)

(21) Appl. No.: 16/080,011

(22) PCT Filed: Jan. 30, 2017
(86) PCT No.: PCT/US17/15691

§ 371 Date: Aug. 26, 2018

Related U.S. Application Data
(60) Provisional application No. 62/288,242, filed on Jan. 28, 2016.

(51) Int. Cl. A61B 34/00; B25J 9/16; B25J 13/02; B25J 13/08

(52) U.S. Cl. CPC A61B 34/76; B25J 9/1689; A61B 2034/302; B25J 13/025; B25J 13/085; B25J 9/1694

(57) ABSTRACT

A minimally invasive medical system comprises a manipulator having a plurality of joints, each of the plurality of joints including a torque and/or force sensor. The manipulator includes an effector configured to receive a surgical instrument. The system comprises a programmable computing device programmed for moving the surgical instrument while estimating surgical forces applied to the patient by the surgical instrument using torque and/or force measurements from the plurality of torque and/or force sensors located at the joints.

This application claims the benefit of U.S. Provisional Application No. 62/288242, which is incorporated herein by reference.

### FIELD OF THE INVENTION

The invention relates generally to the field of robotic surgical systems, and more particularly to systems and methods for estimating forces exerted by a surgical instrument onto tissue of a patient.

### BACKGROUND

As described in U.S. Patent Publications 2010/0094312 and 2013/0012930 (the '312 and '930 applications), incorporated herein by reference, the ability to understand the forces that are being applied to the patient by the surgical tools during minimally invasive surgery is highly beneficial. A determination of the forces imparted to tissue by the tips of the instruments, as well as a determination of the forces applied by the shaft of the instrument to the trocar at the entrance point (or incision) to the body are particularly useful. Furthermore, to minimize tissue trauma at the instrument insertion site (incision), the motion of the instrument shaft and trocar during robotic manipulation of the instrument should be controlled to avoid lateral motion of the shaft at the insertion point, since lateral motions would put extra stress and force on patient tissue at the incision site. Moreover, pivotal motion of the shaft should occur relative to a fulcrum or pivot point located at the insertion point. Understanding the forces applied to the robotically manipulated instrument enables the operator to better control the instrument during surgery while also enabling the control system of the robotic surgical system to determine the location of the fulcrum point and to manipulate the instrument relative to that fulcrum point so as to minimize incision site trauma.

The previously mentioned published patent applications describe the use of a 6 DOF force/torque sensor attached to the robotic manipulator as a method for determining the haptic information needed to provide force feedback to the surgeon at the user interface. They describe a method of force estimation and a minimally invasive medical system, in particular a laparoscopic system, adapted to perform this method. As described, a robotic manipulator has an effector unit equipped with a six degrees-of-freedom (6-DOF or 6-axes) force/torque sensor. The effector unit is configured for holding a minimally invasive instrument mounted thereto. In normal use, a first end of the instrument is mounted to the effector unit and the opposite, second end of the instrument (e.g. the instrument tip) is located beyond an external fulcrum (pivot point kinematic constraint) that limits the instrument in motion. In general, the fulcrum is located within an access port (e.g. the trocar) installed at an incision in the body of a patient, e.g. in the abdominal wall. A position of the instrument relative to the fulcrum is determined. This step includes continuously updating the insertion depth of the instrument or the distance between the (reference frame of the) sensor and the fulcrum. Using the 6 DOF force/torque sensor, a force and a torque exerted onto the effector unit by the first end of the instrument are measured. Using the principle of superposition, an estimate of a force exerted onto the second end of the instrument based on the determined position is calculated.

The present application describes a system capable of carrying out the methods described in the referenced application making use of a plurality of torque and/or force sensors disposed at the joints of the robotic manipulator rather than the 6 DOF force/torque sensor discussed in the referenced applications.

### BRIEF DESCRIPTION OF THE DRAWINGS

FIGS. 1 and 2 show first and second embodiments, respectively, of robotic manipulator arms.

### DETAILED DESCRIPTION

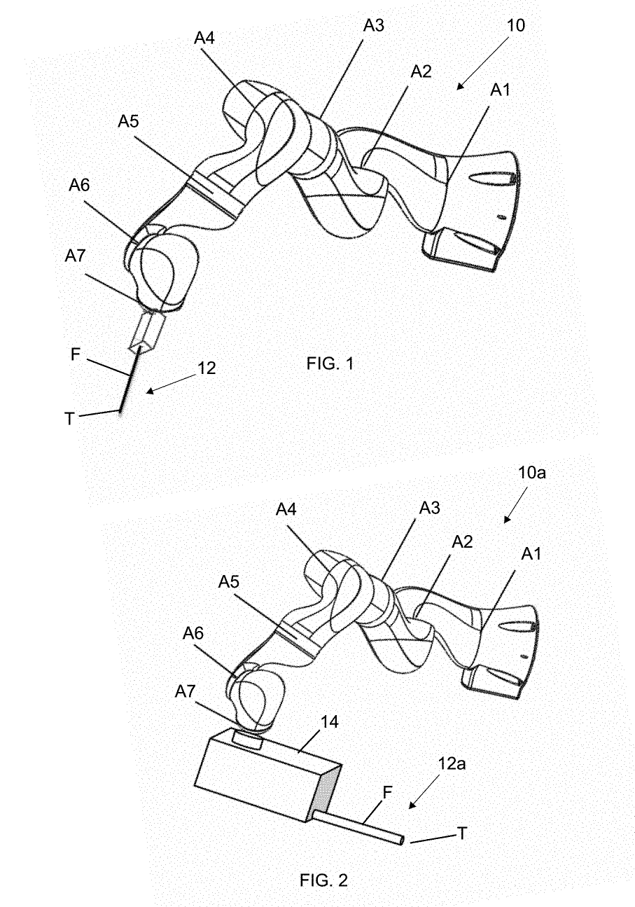

FIG. 1 illustrates a first embodiment of a robotic manipulator 10 which may be supported by a cart, or mounted to the floor, ceiling or patient bed. A surgical instrument 12 (which may be a laparoscopic type of instrument) is mounted to a manipulator end effector unit of the manipulator 10 as shown. The manipulator is part of a surgical system which additionally includes a manipulator controller (not shown) comprising a computer programmed with software for operating one or more such manipulators 10 based on surgeon input received from a surgeon console. The surgeon console includes input devices (e.g. hand controls) manipulated by the surgeon to move the instruments supported by the manipulator. These controls may include hand controls that provide haptic interface for force-feedback to the surgeon corresponding to forces encountered by the instruments 12.

The manipulator consists of multiple degrees of freedom which in this example are shown as seven rotational axes of a robotic arm. More particularly, the manipulator 10 includes a plurality of segments, each rotatable at a joint about a rotation axis. In the illustrated embodiment, the manipulator 10 includes seven such joints and corresponding rotation axes. These are labeled Axis 1 through Axis 7 in the drawings.

A plurality of the joints, which may be each joint, includes sensors such as angular position sensors and/or torque sensors. The external loads applied to the instrument can be determined by using the measured torques and positions at each such joint, adjusting for the known effects of gravity and accelerations. Because the gravity forces and acceleration forces on the joint torque sensors are known given the mass of the payload (the instrument 12) and components of the manipulator 10, and the position of all components of the manipulator and instrument are measured by position sensors, the external loads applied to the instrument can be determined using the total measured torques at each joint. In this case, the torques on each joint, along with the position of each joint are used to calculate the forces and torques being applied to the instrument tip or end effector or the shaft at the incision site. The torque measurements on each of the plurality of degrees of freedom and the position measurements of each such degree of freedom are used to calculate the forces and torques on the instrument tips or at the incision site. This information can also be used to calculate the location of the laparoscopic incision site to ensure that the movement of the robotic manipulator moves the instrument relative to the fulcrum F to avoid trauma at the incision point. The robotic manipulator may have rotational degrees of freedom, translational degrees of freedom, or a combination of the two. In a modified version of the FIG. 1 embodiment, the manipulator arm includes one or more prismatic joints and force sensors are used in place of torque sensors at one or more of the prismatic joints. The robotic manipulator may have any number of degrees of freedom with 1 or more axis including position and force or torque sensing.

In use, an instrument 12 attached to the manipulator 10 is inserted through the incision (or a trocar within the incision). At a point in the procedure when either no forces or well-known forces are applied at the instrument end effector (i.e. after the instrument has been manually inserted into the patient and the surgical personnel have removed hands from the instrument or manipulator), this measurement and calculation method can be used to measure the forces and torques from the patient incision site on the instrument and to determine the position of the patient incision site (using small lateral manipulations of the instrument relative to the incision) to set the location of the fulcrum F to be maintained by the manipulator as it moves robotically during the procedure. During the operation, the forces applied by the instrument end effector can be measured and used to provide haptic feedback to the operator via the surgeon console.

FIG. 2 shows a second embodiment of a manipulator 10a used for a multiple instrument system, in which multiple instruments are deployed through a single trocar 12a is shown. In this embodiment, the robotic manipulator 10a may be attached to a robotic engine 14 (which is also attached to the trocar) housing actuators such as motors used to control one or more of the instruments inside the patient. Just as in the embodiment shown in FIG. 1, the joint position and torque sensors in axes 1-7 provide enough information to determine the fulcrum point that should be maintained by the manipulator during a procedure to minimize trauma at the patient incision site. At a point in the procedure when either no forces or well-known forces are applied at the end effector, this measurement and calculation method can be used to measure the forces and torques from the patient incision site on the trocar and determine the position of the patient incision site. During the procedure, the manipulator can then maintain this point fixed. As with the first embodiment, some of these torque sensors may be replaced by force sensors for a prismatic joint in the manipulator arm that might be used instead of a rotational joint.

What is claimed is:

1. A minimally invasive medical system comprising a manipulator having a plurality of joints, each of the plurality of joints including a torque and/or force sensor, the manipulator further including an effector configured to receive a surgical instrument, said instrument, when held by said effector unit, having a first end mounted to said effector unit, a second end positionable through an incision and within a patient, and a longitudinal axis, the system comprising a programmable computing device programmed for moving said surgical instrument while estimating surgical forces applied to the patient by the surgical instrument using torque and/or force measurements from the plurality of torque and/or force sensors.

2. The system of claim 1, wherein the programmable computing device is programming for estimating surgical forces in the form of forces and/or torques applied by the instrument shaft to the incision site at the instrument or trocar entry point into the abdomen using the position and force or torque measurements on one or more axes of the robotic manipulator.

3. The system of claim 1, wherein the programmable computing device is programming for estimating surgical forces in the form of forces and/or torques applied by the instrument tips to the tissue inside the patient's body, using the position and force or torque measurements on one or more axes of the robotic manipulator.

4. The system of claim 3, wherein the programmable computing device provides input of the estimated surgical forces to a haptic user interface to enable haptic or force feedback to be delivered to an operator.

5. The system of claim 2, wherein the programmable computing device provides input of the estimated surgical forces to a haptic user interface to enable haptic or force feedback to be delivered to an operator.

6. The system of claim 1, wherein the programmable computing device uses the position and force or torque measurements from one or more axes of the robotic manipulator to determine the position of the instrument or trocar entry point into the abdomen of the patient for the purposes of setting the remote center of motion to be maintained by the robotic manipulator to minimize tissue damage at the incision site.

7. The system of claim 1, wherein the surgical instrument comprises a manipulator mounted trocar having a plurality of instruments extending through it.

8. The system of claim 1, wherein a plurality of the joints are rotational joints.

9. The system of claim 8, wherein at least one of the joints is a prismatic joint and wherein the sensor at the prismatic joint comprises a force sensor.

10. A method comprising:
providing a minimally invasive medical system comprising a manipulator having a plurality of joints, each of the plurality of joints including a torque and/or force sensor, the manipulator further including an effector;
mounting a first end of a surgical instrument to the end effector of the manipulator and inserting a second end of the instrument through an incision and into a patient, the instrument including a longitudinal axis,
estimating surgical forces applied to the patient by the surgical instrument using torque and/or force measurements from the plurality of torque and/or force sensors.

11. The method of claim 10, wherein the estimating step includes estimating surgical forces in the form of forces and/or torques applied by the instrument shaft to the incision site at the instrument or trocar entry point into the abdomen using the position and force or torque measurements on one or more axes of the robotic manipulator.

12. The system of claim 10, wherein the estimating step includes estimating surgical forces in the form of forces and/or torques applied by the instrument tips to the tissue inside the patient's body, using the position and force or torque measurements on one or more axes of the robotic manipulator.

13. The method of claim 12, further including delivering input representing the estimated surgical forces from the programmable computing device to a haptic user interface to enable haptic or force feedback to be delivered to an operator.

14. The method of claim 11, further including delivering input representing the estimated surgical forces from the programmable computing device to a haptic user interface to enable haptic or force feedback to be delivered to an operator.

15. The method of claim 10 wherein the programmable computing device uses the position and force or torque measurements from one or more axes of the robotic manipulator to determine the position of the instrument or trocar entry point into the abdomen of the patient for the purposes of setting the remote center of motion to be maintained by the robotic manipulator to minimize tissue damage at the incision site.

16. The method of claim 1, wherein the estimating step is performed while robotically moving the instrument within the incision.
